(19) United States
(12) Patent Application Publication
(10) Pub. No.: US 2013/0116765 A1
(43) Pub. Date: May 9, 2013

(54) IMPLANTABLE LEAD ADAPTOR WITH MRI FILTER

(71) Applicant: Oscor Inc., Palm Harbor, FL (US)

(72) Inventors: Thomas P. Osypka, Palm Harbor, FL (US); Nicholas Vidalis, Clearwater, FL (US); Brian Walguarnery, Tampa, FL (US)

(73) Assignee: Oscor Inc., Palm Harbor, FL (US)

(21) Appl. No.: 13/670,842

(22) Filed: Nov. 7, 2012

Related U.S. Application Data
(60) Provisional application No. 61/556,481, filed on Nov. 7, 2011.

(51) Int. Cl. A61N 1/05; B23P 11/00

(52) U.S. Cl. 607/119; 29/401.1

(57) ABSTRACT

A system for use with an implantable lead wire includes an implantable electronic apparatus configured to generate an electrical signal. An implantable lead adaptor is operatively disposed between the proximal end of the implantable lead wire and the implantable electronic apparatus. A band stop filter is housed within the implantable lead adaptor and electrically coupled in series with the implantable lead wire and the implantable electronic apparatus.

### BACKGROUND OF THE INVENTION

1. Field of the Invention

The present invention relates generally to implantable medical device assemblies, and more particularly, to implantable medical device assemblies which operably couple implantable lead wires and medical devices such as pacemakers and the like, while improving their compatibility with MRI technology.

2. Description of Related Art

Implantable electrical leads are utilized in a variety of therapeutic procedures, including neurostimulation for the treatment of chronic pain, electrical sacral nerve stimulation for bladder control, and cardiac pacing and defibrillation for treating arrhythmia. A typical lead consists of one or more conductors extending through an elongated body. The conductors allow for electrical contact between tissue to be stimulated and a remote device which controls electrical stimulation of the tissue. The remote device often includes a signal generator for delivering electrical energy to the lead wire and a diagnostic system for monitoring bodily functions.

Once implanted in a patient, traditional leads prevent or significantly limit a patient's ability to be scanned with magnetic resonance imaging (MRI) on account of the dangers presented by powerful magnetic fields created by MRI scanners. Such magnetic fields can have harmful interactions with metallic materials used in traditional electrical leads, electrodes, and other metallic structures of the implanted leads. For example, MRI scanners may induce undesirable voltages and electromagnetic interference (EMI) signals in an implanted lead wire system. The resulting undesirable currents produced can be sufficient to heat and destroy adjacent tissue.

In addition to their general incompatibility with MRI scanners and the like, standard implantable leads may be also be incompatible with newer implantable medical devices. This may necessitate invasive surgery to remove and replace the existing lead with a more compatible one to facilitate implantation of a new device. Improving the compatibility of implanted leads with various medical devices and technologies such as MRI scanning can improve a patient's medical and preventative care while reducing the patient's risk, time, and medical costs associated with such care.

Such conventional methods and systems which include lead wires and associated medical devices have generally been considered satisfactory for their intended purposes. However, there is still a need in the art for improving the compatibility of implantable lead wires with MRI technology and the like. The present invention provides a solution for these problems.

### SUMMARY OF THE INVENTION

The invention includes a system for use with an implantable lead wire having proximal and distal ends. The system includes an implantable electronic apparatus such as a pulse generator or defibrillator which is configured to generate an electrical signal, an implantable lead adaptor operatively disposed between the proximal end of the lead wire and the electronic apparatus, and a band stop filter mechanically coupled to the lead adaptor and electrically coupled to both the lead wire and the electronic apparatus.

In certain embodiments, the lead adaptor is separate and distinct from the lead wire and the electronic apparatus. The lead adaptor has a proximal end configured to detachably couple to the electronic apparatus, and a distal end configured to detachably couple to the proximal end of the lead wire.

In certain embodiments, the lead adaptor houses and positions the band stop filter in a small epoxy compartment in a manner which electrically couples the band stop filter to the lead wire and the electronic apparatus when the proximal and distal ends of the lead adaptor are detachably coupled to the electronic apparatus and lead wire respectively.

In accordance with certain embodiments, the band stop filter includes a capacitor and an inductor selected such that the band stop filter is resonant at a selected frequency. The capacitor is configured in parallel with an inductor, and the capacitor and inductor are electrically coupled in series with the implantable lead wire and the implantable electronic apparatus. The Q factor of the inductor may be relatively maximized and the Q factor of the capacitor may be relatively minimized to reduce the overall Q value of the band stop filter, attenuate current flow through the implantable lead wire along a range of selected frequencies, preferably along a pre-selected range of MRI pulsed frequencies. The band stop filter is also preferably separate and distinct from the lead wire and the electronic apparatus, but electrically coupled thereto via detachable coupling of the lead adaptor as discussed above.

The band stop filter functions to abate or eliminate undesirable and harmful voltages and electromagnetic interference signals and currents which can be produced in the implanted lead wire by MRI pulsed frequencies and the like. It will be appreciated by those skilled in the art that the arrangement of the lead adaptor and the band stop filter proximal of the lead wire and distal to the electronic apparatus facilitates easy replacement of the electronic apparatus, lead adaptor, and/or band filter without requiring invasive surgical procedures to replace the already implanted lead wire.

A method in accordance with the present invention includes decoupling an electronic apparatus from an implanted lead wire, providing an adaptor which includes a band stop filter, and mechanically coupling the adaptor to the implanted lead wire and the electronic apparatus such that the band stop filter is electrically coupled to and operatively disposed between the implanted lead wire and the electronic apparatus.

For example, if a given band stop filter is old, broken, or defective, then a different band stop filter having the same overall Q value may be implemented by swapping out the existing lead adaptor (which will often be located within the patient at an easy-to-reach (e.g., proximal, superficial location such as near the surface of the skin) with a replacement lead adaptor that houses the same band stop filter. In another example, if a band stop filter having a different Q value is desired (to render the existing implanted lead wire compatible with a different scanning technology or MRI setting), then a band stop filter with the desired Q value may be implemented by similarly swapping out the existing lead adaptor with a new one that houses a band stop filter having the desired Q value.

It will be appreciated that the lead adaptor can be easily replaced with a different lead adaptor having the same type of distal end, a different type of proximal end, and the same type of band stop filter housed within it. Such a replacement would render the existing implanted lead wire compatible with a new or different pulse generator, defibrillator, or other electronic device (by interfacing the new or different device to the new proximal end of the replacement lead adaptor, thereby electrically coupling the new or different device to the existing lead wire) while maintaining MRI compatibility. Importantly, none of these procedures require surgical removal or replacement of the existing lead wire to access or replace the existing lead adaptor or band stop filter.

These and other features of the system and methods of the subject invention will become more readily apparent to those skilled in the art from the following detailed description of the preferred embodiments taken in conjunction with the drawings.

### BRIEF DESCRIPTION OF THE DRAWINGS

So that those skilled in the art to which the subject invention pertains will readily understand how to make and use the devices and methods of the subject invention without undue experimentation, preferred embodiments thereof will be described in detail herein below with reference to certain figures, wherein:

FIG. 1 is an exploded perspective view of an exemplary embodiment of a system for use with an implantable lead wire in accordance with the present invention, showing an implantable electronic apparatus, an implantable lead adaptor, and a lead wire;

FIG. 2 is a perspective view of a portion of the system of FIG. 1, showing the lead wire, lead adaptor, and electronic apparatus connected together;

FIG. 3 is a schematic view of the implantable lead adaptor of FIG. 1 equipped with and housing a band stop filter; and

FIG. 4 is a schematic view of the circuit of the band stop filter of FIG. 3, showing the parallel capacitor and inductor.

### DETAILED DESCRIPTION OF THE PREFERRED EMBODIMENTS

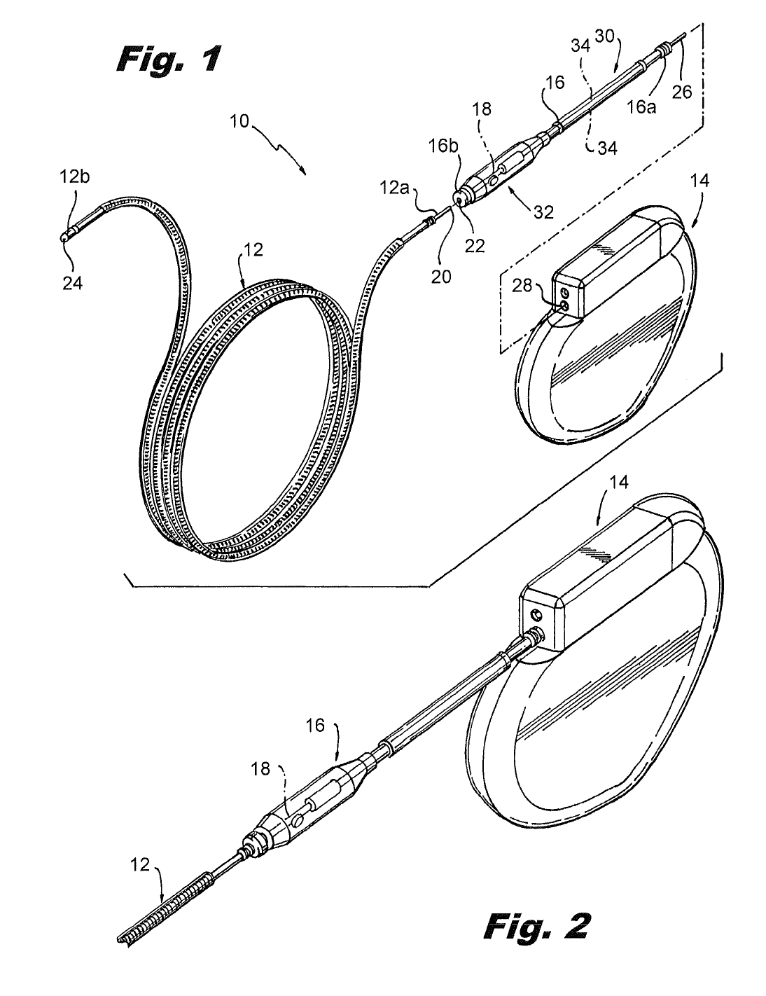

Reference will now be made to the drawings wherein like reference numerals identify similar structural features or aspects of the subject invention. For purposes of explanation and illustration, and not limitation, the exemplary embodiment of the system in accordance with the invention is shown in FIG. 1 and is designated generally by reference character 10.

Turning now to FIGS. 1 and 2, a system 10 for use with an implantable lead wire 12 is provided. The system 10 includes an implantable electronic apparatus 14 such as a pulse generator or defibrillator which is configured to generate an electrical signal, an implantable lead adaptor 16 operably disposed between the lead wire 12 and the electronic apparatus 14 for mechanically and electrically coupling the lead wire 12 to the electronic apparatus 14, and a band stop filter 18 mechanically coupled to the lead adaptor 16 for attenuating current flowing through the lead wire 12 along a range of selected frequencies.

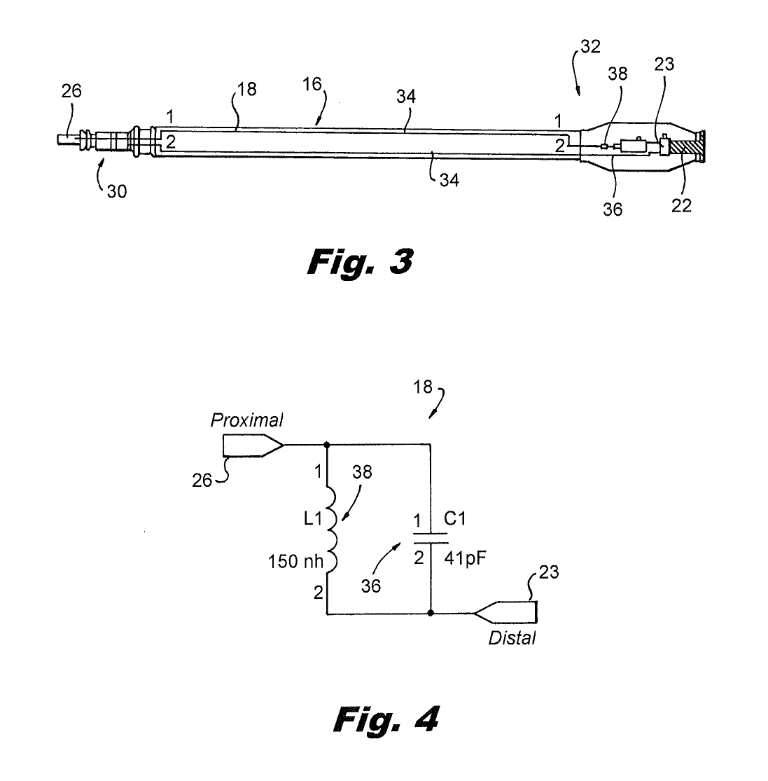

The lead wire 12 extends between a proximal end 12a and a distal end 12b, and has at least one electrical conductor extending through an interior passage of the lead wire 12. The lead wire 12 may be of any type known in the art for pacing, defibrillation, neurostimulation, or other uses, such as, by way of example, those disclosed in U.S. Patent Application Publication No. 2006/0095107 to Osypka; U.S. Pat. No. 7,197,361 to Van den Nieuwenhof et al; and U.S. Pat. No. 6,978,185 to Osypka, each of which is incorporated by reference herein in its entirety. The lead wire 12 includes a proximal lead connector 20 (e.g. IS-1, IS-4, etc.) which is traditionally used to connect directly to an electronic apparatus such as a pulse generator or defibrillator. In the preferred embodiment shown in FIG. 1, the proximal lead connector 20 connects to the lead adaptor 16 through an aperture or lumen 22 defined by the lead adaptor 16, thereby electrically coupling the lead wire 12 to the band stop filter 18 and electronic apparatus 14. The lead wire 12 also includes a distal lead tip 24 which houses one or more electrodes used for sensing and imparting electrical impulses to tissue of a patient targeted for treatment or stimulation. The implantable lead adaptor 16 may be constructed similar to conventional lead adaptors known in the art, such as, by way of example, those disclosed in U.S. Pat. No. 7,270,568 to Osypka, which is herein incorporated by reference in its entirety. The proximal end 16a of the lead adaptor 16 includes a proximal connector 26 configured to connect to a receptacle portion or lumen 28 defined by the electronic apparatus 14. The distal end 16b of the lead adaptor 16 defines the adapter lumen 22 which operably receives the proximal lead connector 20 of the lead wire 12. The adapter lumen 22 is sized to accommodate the proximal lead connector 20 of a standard lead wire, but can be sized to accommodate lead wires of varying shapes, sizes, types, and contact configurations, such as, for example, unipolar or bipolar IS-1, LV-1 and DF-1 type lead connectors. The lead adaptor 16 includes a proximal housing portion 30 and a distal housing portion 32. The proximal housing portion 30 insulates and houses at least a portion of the proximal connector 26, as well as at least a proximal portion of wires 34 which are electrically coupled to the proximal connector 26 and extend through the lead adaptor 16 to the distal end 16b thereof. The distal housing portion 32 houses the primary components of the band stop filter 18, namely a capacitor 36 and an inductor 38 (FIG. 3), as well as a distal portion of the wires 34. The housing portions 30, 32 are preferably formed from Epoxy or similar materials which are biocompatible and have a high electrical insulation resistance. The housing portions 30, 32 could also be made from other suitable materials such as silicone or polyurethane. In the assembled configuration of FIG. 2, the lead adaptor 16 and band stop filter 18 are operably disposed between the electronic apparatus 14 and the lead wire 12 with the adaptor 16 mechanically coupled to both the lead wire 12 and the electronic apparatus 14, and the band stop filter 18 electrically coupled to both the lead wire 12 and the electronic apparatus 14. The proximal lead connector 20 of the lead wire 12 shown in FIG. 1 is fully inserted into the lumen 22 defined by the adaptor 16 and mechanically coupled thereto interference fit or by any other suitable type of engagement. Similarly, the proximal connector 26 of the adaptor 16 is fully inserted into the lumen 28 defined by the electronic apparatus 14, and mechanically coupled to the electronic apparatus 14 by any suitable type of engagement.

Turning now to FIGS. 3 and 4, the band stop filter 18 is incorporated into the housing portions 30 and 32 with the capacitor 36 and inductor 38 housed within the distal portion 32 thereof. The proximal housing portion 30 of the adaptor 16 houses at least a portion of the proximal connector 26, as well as at least a proximal portion of the wires 34 of the band stop filter 18. As shown in FIG. 4, the capacitor 36 and inductor 38 are electrically wired in parallel relative to each other but in series with the proximal connector 26 and a distal contact 23 adjacent the lumen 22. Thus, when the lumen 22 of the adaptor 16 operably receives the proximal lead connector 20 of the lead wire 12, and when the proximal connector 26 of the adaptor 16 is inserted into the lumen 28 of the electronic apparatus 14, the band stop filter 18 is electrically coupled to the lead wire 14 and the electronic apparatus 14, and operatively disposed therebetween. It will be appreciated that other types of filters may alternatively or additionally be utilized.

The band stop filter 18 functions to abate or eliminate the undesirable and harmful voltages and electromagnetic interference signals and currents produced in the implanted lead wire 12 arising from MRI or other sources of EMI. The capacitance value of the capacitor 36 and inductance value of the inductor 38 are selected on an application to application basis such that the band stop filter 18 is effective to filter out one or more selected frequencies, and preferably along a range of MRI pulsed frequencies. The Q factor of the inductor 38 may be relatively maximized and the Q factor of the capacitor relatively minimized to reduce the overall Q value of the band stop filter 18 to attenuate current flow through the implanable lead wire 12 along the range of selected frequencies.

Use of band stop filter in association with a a lead wire is disclosed in U.S. Pat. No. 7,363,090 to Halperin et al (hereinafter, ‘Halperin’), which is herein incorporated by reference in its entirety. Halperin discloses use of a band stop filter along the length of a lead wire between its proximal and distal ends to limit MRI induced current therethrough. The present invention improves upon this disclosure by operatively situating the band stop filter 18 proximal of the lead wire 14, and by housing the band stop filter 18 in a lead adaptor 16 which mechanically couples to the proximal end 12a of the lead wire 12.

It will be appreciated by those skilled in the art that the arrangement of the lead adaptor 16 and the band stop filter 18 proximal of the lead wire 12 and distal to the electronic apparatus 14 as disclosed herein facilitates easy replacement of the electronic apparatus 14, lead adaptor 16, and/or band filter 18 without requiring invasive surgical procedures to remove or replace the lead wire 12. For example, if a given band stop filter is old, broken, or defective, then a different band stop filter having the same overall Q value may be implemented by swapping out the existing lead adaptor with a replacement lead adaptor that houses the same band stop filter.

Alternatively, if a band filter having a different Q value is desired (to render the existing implanted lead wire compatible with a different scanning technology or MRI setting), then a band filter with the new Q value may be implemented by similarly swapping out the existing lead adaptor with a new one that houses a band filter having the desired Q value. All of these procedures can be completed without removing the lead wire 12.

While the lead adaptor 16 disclosed herein is preferably separate and distinct from the lead wire 12 and the electronic apparatus 14 and detachably coupled thereto, it will be appreciated that the lead adaptor 16 could also be permanently incorporated into the proximal end of a lead wire or the distal end of an electronic apparatus with the band stop filter 18 housed within the adapter 16 as described herein. Also, while the band stop filter 18 is shown housed within the lead adaptor 16, it will be appreciated that the band stop filter 18 could also be operably disposed partly or completely external to the lead adaptor 16, or electrically coupled to the lead wire 12 proximal of the lead wire 12 without use of an adaptor 16.

It will also be appreciated that the lead adaptor 16 can be easily replaced with a different lead adaptor having the same type of distal end, a different type of proximal end, and the same type of band stop filter housed within it. Such a replacement would render the existing implanted lead wire compatible with a new or different pulse generator, defibrillator, or other electronic device (by interfacing the new or different device to the new proximal end of the replacement lead adaptor, thereby electrically coupling the new or different device to the existing lead wire) while maintaining MRI compatibility. As described above, these procedures can be completed without removal of the implanted lead wire 12.

The systems and methods of the present invention, as described above and shown in the drawings, provide for an improved system for use with an implantable lead wire, the system improving the compatibility of existing lead wires with various electronic apparatuses while improving and maintaining MRI compatability and reducing associated medical costs and risks. While particular lead wires, electronic apparatuses, and lead adaptors have been disclosed, it will be appreciated that various other types of lead wires, electronic apparatuses, and lead adaptors may additionally or alternatively be utilized. While a particular band stop/band pass filter circuit has been disclosed, it will be appreciated that other band stop filter circuits or other filters may alternatively or additionally be utilized for attenuating signals along a selected range of frequencies. While particular materials and shapes have been disclosed for a lead wire and an adaptor, it will be appreciated that other materials and shapes may also be utilized. It will therefore be appreciated that while the apparatus and methods of the subject invention have been shown and described with reference to preferred embodiments, those skilled in the art will readily appreciate that changes and/or modifications may be made thereto without departing from the spirit and scope of the subject invention as claimed.

1. A system for use with an implantable lead wire having a proximal end and a distal end, the system comprising:
a) an implantable electronic apparatus configured to generate an electrical signal;
b) an implantable lead adaptor operatively disposed between the proximal end of the implantable lead wire and the implantable electronic apparatus; and
c) a band stop filter housed within the implantable lead adaptor and electrically coupled in series with the implantable lead wire and the implantable electronic apparatus.

2. A system according to claim 1, wherein the implantable lead adaptor is separate and distinct from the implantable lead wire.

3. A system according to claim 1, wherein the implantable lead adaptor is coupled to the proximal end of the implantable lead wire.

4. A system according to claim 3, wherein the implantable lead adaptor is detachably coupled to the proximal end of the implantable lead wire.

5. A system according to claim 1, wherein the implantable electronic apparatus includes a pulse generator.

6. A system according to claim 1, wherein the implantable electronic apparatus includes a defibrillator.

7. A system according to claim 1, wherein the band stop filter has an overall Q value selected to attenuate current flow through the implantable lead wire along a range of MRI pulsed frequencies.

8. A system according to claim 1, wherein the band stop filter has a capacitor configured in parallel with an inductor, and the capacitor and inductor are electrically coupled in series with the implantable lead wire and the implantable electronic apparatus.

9. A system according to claim 1, wherein the band stop filter has a capacitor and an inductor configured in parallel with each other and in series with the lead wire.

10. A system according to claim 9, wherein the implantable lead adaptor is coupled to the proximal end of the implantable lead wire and to the implantable electronic apparatus.

11. A system according to claim 10, wherein the band stop filter has an overall Q value selected to attenuate current flow through the implantable lead wire along a range of MRI pulsed frequencies.

12. A system according to claim 11, wherein
b) the band stop filter is configured to electrically couple with the proximal end of the implantable lead wire and the implantable electronic apparatus for attenuating the current flow through the implantable lead wire along the range of MRI pulsed frequencies.

13. (canceled)

14. An adaptor for operatively connecting an implantable lead wire to an implantable electronic apparatus, the adaptor comprising:
a) a proximal connector portion configured to connect to the implantable electronic apparatus;
b) a distal connector portion configured to connect to the implantable lead wire; and
c) a band stop filter mechanically and electrically coupled to and operatively disposed between the proximal connector portion and the distal connector portion.

15. A method of preparing an implanted electronic device for MRI scanning, comprising:
a) decoupling an implanted electronic device from an implanted lead wire;
b) providing an adaptor which includes a band stop filter; and
c) mechanically coupling the adaptor to the the implanted lead wire and the electronic apparatus such that the band stop filter is electrically coupled to the implanted lead wire and the electronic apparatus.

16. A method according to claim 15, wherein the band stop filter is housed within the adaptor.

17. A method according to claim 15, wherein the band stop filter is configured and adapted to attenuate current flow through the implanted lead wire along a range of MRI pulsed frequencies.

18. An adaptor according to claim 14, wherein the implantable electronic apparatus includes a defibrillator.

19. An adaptor according to claim 14, wherein the implantable electronic apparatus includes a pulse generator.

20. An adaptor according to claim 14, wherein the band stop filter is configured and adapted to attenuate current flow through the implanted lead wire along a range of MRI pulsed frequencies.
